(19) United States
(12) Patent Application Publication
(10) Pub. No.: US 2004/0053219 A1
(43) Pub. Date: Mar. 18, 2004

(54) Method for analysing human immunodeficiency virus (HIV) phenotypic characteristics

(75) Inventors: Francois Clavel, Paris (FR); Esther Race, Montrouge (FR); Veronique Obry, La Grarenne Colombes (FR); Fabrizio Mammano, Paris (FR); Elisabeth Dam, Paris (FR); Virginie Trouplin, Paris (FR)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(73) Assignees: Bioalliance Pharma (S.A.); Inserm

(21) Appl. No.: 10/436,458

(22) Filed: May 13, 2003

Related U.S. Application Data
(63) Continuation of application No. 09/817,135, filed on Mar. 27, 2001, now abandoned.

(30) Foreign Application Priority Data
Nov. 10, 2000 (FR) 00/14495
Mar. 23, 2001 (FR) 01/03970

(51) Int. Cl. C12Q 1/70; C12P 19/34

(52) U.S. Cl. 435/5; 435/91.2

(57) ABSTRACT

The present invention relates to a method for analysing the phenotypic characteristics shown by certain virus strains, particularly human immunodeficiency viruses, involving the construction of a recombinant virus obtained by homologous recombination.

The present invention also relates a kit comprising the primers, vectors, cell hosts, products and reagents required to carry out PCR amplification, and the products and reagents used to detect a marker, for the implementation of the method according to the invention.

The present invention relates to a method for analysing the phenotypic characteristics shown by certain virus strains, particularly HIV virus.

Genotype tests which are rapid and widely available to detect the presence of mutations in viral genes have been developed for example to detect mutations present in the genes coding for HIV protease and reverse transcriptase.

Although some mutations have been associated with a specific viral activity inhibitor, many others are associated with treatment with several molecules. In addition, with the development of new inhibiting molecules, genotyping of virus variants which escape treatments is becoming increasingly complex. This makes it difficult to perform an evaluation and know to which inhibitors the viruses have become resistant or have retained a slight susceptibility. Under these circumstances, the presence and accumulation of resistance mutations can surely give a good indication of the progression of resistance, but simply detecting these mutations on a genotypic level is no longer sufficient to evaluate the level of resistance quantitatively; this parameter is crucial for optimising therapeutic orientations for patients having undergone failed antiviral therapy.

The phenotypic characteristics of the viruses are related to the various aspects of viral behaviour and directly involved in numerous interactions between said viruses and their environment.

Only phenotype tests, which measure directly in the culture medium the modification of the phenotypic characteristic of the virus, such as viral activity inhibition, for example in the presence of compounds inhibiting said viral activity, provide a quantitative indication of resistance.

Other phenotypic characteristics for which analysis is of interest, particularly from a medical point of view, are those which give a virus its resistance to inhibiting agents liable to inhibit at least one mechanism involved in viral activity, those which give a virus its replicative capacity, those which give a virus is tropism to particular targets or those which give a virus its ability to be neutralised by molecules, such as antibodies, chemokines or inhibitors.

The phenotypic characteristics of HIV virus for which analysis is of particular interest from a medical point of view include those related to the expression of genes, liable to undergo at least one mutation, located in the GAG, ENV, or POL regions of the viral genome, such as those listed below:

I. Infectivity, Replicative Capacity and Virulence.

These phenotypic characteristics of HIV viruses may be related to the function of all the regions of the viral genome, for parts coding for proteins or regions involved in the different mechanisms or steps of the viral replication cycle. In particular, it is important to evaluate the effect produced by mutations in the genes coding for protease, reverse transcriptase, integrase or envelope on viral replication, especially in viruses having developed a resistance to antiviral agents.

Its analysis is used to measure the replicative capacity of a virus, also known as the infectivity or “fitness”.

II. Susceptibility/Resistance to Reverse Transcriptase Inhibitors.

This phenotypic characteristic of HIV viruses is related to the expression of a part of the POL region coding for reverse transcriptase.

Its analysis is used to adjust anti-retroviral treatments with nucleoside analogues or non-nucleoside reverse transcriptase inhibitors (NNRTI).

III. Susceptibility/Resistance to Integrase Inhibitors.

This phenotypic characteristic of HIV viruses is related to the expression of a part of the POL region coding for integrase.

Its analysis provides indications for adjusting anti-retroviral treatments with integrase inhibitors.

IV. Susceptibility/Resistance to Target Cell Virus Entry Inhibitors.

This phenotypic characteristic of HIV viruses is related to the expression of HIV envelope glycoprotein, particularly the expression of the transmembrane sub-unit of said glycoprotein coded by a part of the ENV gene.

The analysis of this phenotypic characteristic is used to demonstrate the action of inhibiting agents which inhibit the fusion of the viral membrane with the target cell membrane.

V. Susceptibility/Resistance to Inhibitors Targeting HIV Virus Co-Receptors.

This phenotypic characteristic is also related to the expression of at least part of the ENV region of HIV viruses which codes for polypeptides which take part in the bond with target cell co-receptors. The envelope/co-receptor interaction enables the entry of the HIV virus into said target cell.

Its analysis is used to measure the resistance of HIV viruses to the action of inhibitors inhibiting the co-receptors used by HIV to enter the target cell.

The inhibiting agents interfere with the co-receptors by inhibiting its interaction with the HIV envelope.

In particular, it is important to evaluate the effect of mutations in the ENV region which modify the interaction of certain regions of the envelope protein with the CXCR4 or CCR5 receptors of the HIV target cells.

VI. Tropism

This phenotypic characteristic is also related to the expression of at least part of the ENV region coding for HIV virus envelope polypeptides, which take part in the bond with one or more target cell receptors, particularly with CXCR4 or CCR5 co-receptors.

Its analysis is used to measure in vivo the HIV virus's capacity to use said receptors, particularly CXCR4 or CCR5, which are expressed differently in various cell types and indicates whether the virus uses either of the receptors, or both.

The information obtained from this analysis can be used to deduce the viral behaviour in certain types of cells, natural HIV targets.

VII. Virulence

This phenotypic characteristic is also related to the expression of all of part of the ENV region which codes for HIV virus envelope polypeptides.

Its analysis is used to evaluate the cytopathogenic power of an HIV virus.

VIII. Neutralising Capacity.

This phenotypic characteristic is also related to the expression of HIV virus envelope proteins.

Its analysis is used to evaluate the susceptibility of viruses to the inhibitory action of antibodies or substances naturally present in the body and present in serum or other fluids.

The first tests to detect for example the phenotypic characteristic of HIV virus resistance to antiviral treatments were performed using primary isolates and peripheral blood lymphocytes (PBL) stimulated with phytoagglutinin (PHA) according to a laborious procedure that is difficult to reproduce. An innovative alternative to these tests, a recombinant virus test, hereafter referred to as RVA was proposed by Kellam and Larder in 1994.

This RVA analytical method measured the resistance of a recombinant virus comprising reverse transcriptase isolated from the plasma from a patient carrying the virus by co-transfection of sequences of said virus duly amplified using a polymerisation chain reaction (PCR), with a virus clone obtained in the laboratory, in which its reverse transcriptase is deleted and which is competent for replication in a variety of well-established cell lines.

Several modifications to this method have now been disclosed (Boucher, C., Keulen, W., Bommel, T., Nijhuis, M., Jong, D., Jong, M., Schopper, P. and Back, N., K (1996) “HIV-1 drug susceptibility determination by using recombinant viruses generated from patient sera tested in a cell-killing assay. Antimicrobial Agents and Chemotherapy” 40(10), 2404-2109) (Shi, C. and Mellors, J. W. (1997) “A recombinant retroviral system for rapid in vivo analysis of human immunodeficiency virus type 1 susceptibility to reverse transcriptase inhibitors”. Antimicrob Agents Chemother 41(12), 2781-5) (Hertogs, K., de Bethune, M. P., Miller, V., Ivens, T., Schel, P., Van Cauwenberge, A., Van Den Eynde, C., Van Gerwen, V., Azijn, H., Van Houtte, M., Peeters, F., Staszewski, S., Larder, B. and Pauwels, R. (1998) “A rapid method for simultaneous detection of phenotypic resistance to inhibitors of protease and reverse transcriptase in recombinant human immunodeficiency virus type 1 isolates from patients treated with antiretroviral drugs”, Antimicrob Agents Chemother 42(2), 269-76.) (Hecht, F. M., Grant, R. M., Petropoulos, C. J., Dillon, B., Chesney, M. A., Tian, H., Hellmann, N. S., Bandrapalli, N. I., Digilio, L., Branson, B. and Kahn, J. O. (1998) “Sexual transmission of an HIV-1 variant resistant to multiple reverse-transcriptase and protease inhibitors”. N. Engl J Med 339(5), 307-11) (Medina, D. J., Tung, P. P., Nelson, C. J., Sathya, B., Casareale, D. and Strair, R. K. (1998) “Characterization and use of a recombinant retroviral system for the analysis of drug resistant HIV”. J Virol Methods 71(2), 169-76).

However, most of these recombinant systems involve disadvantages since, as for the method using PBMC, the production of a reserve of infectious particles expressing a specific phenotypic characteristic to be detected and measured requires an amplification of the virus by exponential growth of lymphocyte cells. The virus is then subjected to genetic deviations during its replication and may lose mutations which are essential for the expression of the phenotypic characteristic to be detected thus modifying the reliability of the method.

Another disadvantage to detect for example a phenotypic characteristic of resistance which is present in analytical methods of the prior art stems from the fact that the simultaneous presence of several mutations liable to induce resistance to different retroviral inhibitors reduces the replicative capacity of the virus.

The inventors have developed a new method for analysing a phenotypic characteristic of HIV viruses which only requires a single replication cycle.

This analytical method is based on the construction of a recombinant virus (RAV) obtained by co-transfection and homologous recombination with:

a) the DNA sequences obtained from an HIV under analysis liable to comprise mutations liable to modify the phenotypic characteristic to be detected, said sequences being extracted from a biological medium such as plasma, serum, saliva, semen or other secretions, from a patient carrying said virus,

b) a first vector comprising a specific deletion of the sequences enabling the replication of HIV and a deletion of all or part of the sequence giving the HIV the phenotypic characteristic to be detected and

c) a second vector, for example, a plasmid, comprising the sequences completing those required for the replication of said virus and which are absent from the first vector.

The method developed by the inventors is rapid, it requires approximately seven days to be carried out and can therefore be used for routine determinations such as the measurement of the susceptibility of patients infected with HIV to viral activity inhibitors.

In this way, the inventors also disclosed in the patent U.S. Pat. No. 6,103,462 a first application of this analytical method, based on the formation of a particular recombinant virus, to determine the susceptibility of an HIV virus to protease inhibitors.

The new analytical methods implemented according to the invention are based on the determination of HIV virus phenotypic characteristics associated with mutations liable to be present at least in one gene chosen from the group comprising gag, pol, protease, reverse transcriptase, RNAse H, integrase, vif, vpr, tat, rev, vpu, env, nef, cis-active sequences, LTR, dimerisation sequences, splicing regulating sequences, RRE by means of ad hoc recombinant viruses.

Therefore, the invention relates to a method for analysing a phenotypic characteristic of HIV viruses present in a biological specimen from a patient, said phenotypic characteristic resulting from one or more mutations of the viral genome liable to influence the viral infection, characterised in that it comprises:

a) the extraction of the nucleic acids contained in a biological specimen,

b) at least one PCR amplification of a segment of the nucleic acids from step a, each with a pair of primers bordering a nucleic acid sequence of the viral genome liable to comprise at least one mutation,

c) the preparation of a vector comprising the parts of an HIV virus genome required for viral replication except for the segment amplified in step b and, if applicable, except for the gene coding for envelope protein,

d) the transfection of a first cell host with:

the nucleic acids contained in step b,

the vector prepared in step c,

if applicable, a second vector comprising a gene coding for an envelope protein if the envelope gene is deleted from the vector prepared in step c,

to obtain a chimeric virus by homologous recombination,

e) the culture of said first cell host under conditions enabling the production of viral particles during a single replication cycle,

f) the infection by the viral particles obtained in step e of at least one second cell host liable to be infected by an HIV virus or an HIV pseudotype virus and comprising, if applicable, a marker gene that can only be activated following viral infection, and

g) the detection and/or quantification of the marker expressed in step f in order to detect at least one characteristic of the HIV viruses present in the biological specimen.

More specifically, the PCR amplification in step b is carried out with a pair of primers bordering a nucleic acid sequence comprising all or part of a viral genome region selected from: gag, pol, protease, reverse transcriptase, RNAse H, integrase, vif, vpr, tat, rev, vpu, env, nef, cis-active sequences, LTR, dimerisation sequences, splicing regulating sequences or Rev response element (RRE).

According to a specific embodiment of the analytical method according to the invention, the PCR amplification in step b is carried out with a pair of primers bordering a nucleic acid sequence coding for a part of the gag protein of the human immunodeficiency virus and a nucleic acid sequence coding for protease, liable to comprise at least one mutation in the gene coding for protease and, the vector from step c is constructed from an HIV virus genome in which all or part of the gene coding for protease is deleted.

Advantageously, the amplification in step b according to the analytical method of the invention, of a nucleic acid sequence liable to comprise at least one mutation in the gene coding for protease is performed with a pair of primers of a size between 10 and 50 oligonucleotides, comprising the sequences Fit A-: (5′ TCA CCT AGA ACT TTA AAT GC 3′) (SEQ ID No: 1) and Pro A-: (5′ GGC AAA TAC TGG AGT ATT GTA TG3′ 3′) (SEQ ID No: 2), or composed of fragments of said sequences, or analogue sequences of said sequences comprising mutations of one or more nucleotides which do not essentially modify their ability to hybridise the region of the protease gene comprising the mutation(s), followed by a second amplification step with a pair of primers of a size between 10 and 50 oligonucleotides, comprising the sequences: Fit B: (5′ AGA ACT TTA AAT GCA TGG GT 3′) (SEQ ID No: 3) and Pro B-: (5′ GGA GTA TTG TAT GGA TTT TCA GG 3′) (SEQ ID No: 4), or composed of fragments of said sequences, or analogue sequences of said sequences comprising mutations of one or more nucleotides which do not essentially modify their ability to hybridise the region of the protease gene comprising the mutation(s).

More preferentially, the amplification in step b according to the analytical method of the invention, of a nucleic acid sequence liable to comprise at least one mutation in the gene coding for protease is performed with a pair of primers:

(SEQ ID No:1)

| | |
|---|---|
| Fit A-: | (5′ TCA CCT AGA ACT TTA AAT GC 3′) and |

(SEQ ID No:2)

| | |
|---|---|
| Pro A-: | (5′ GGC AAA TAC TGG AGT ATT GTA TG3′ 3′), |

followed by a second amplification step with a pair of primers:

(SEQ ID No:3)

| | |
|---|---|
| Fit B: | (5′ AGA ACT TTA AAT GCA TGG GT 3′) and |

(SEQ ID No:4)

| | |
|---|---|
| Pro B-: | (5′ GGA GTA TTG TAT GGA TTT TCA GG 3′), |

to obtain a DNA segment with 1460 base pairs, ranging from the residues 3950 and 5410 inclusive, and the vector from step c is a retroviral vector deleted from the region of the pol reading frame coding for HIV-1 protease ranging from the residues 1505 to 2565 inclusive, deleted from the envelope region and comprising a single MluI restriction site.

According to a second specific embodiment of the analytical method according to the invention, the PCR amplification in step b is carried out with a pair of primers bordering a nucleic acid sequence liable to comprise at least one mutation in the gene coding for reverse transcriptase, and the transfection in step c is carried out with a first vector constructed from an HIV virus genome in which all or part of the gene coding for reverse transcriptase is deleted.

Advantageously, the amplification in step b according to the analytical method of the invention, with a pair of primers bordering a nucleic acid sequence liable to comprise at least one mutation in the gene coding for reverse transcriptase is performed with a pair of primers of a size between 10 and 50 oligonucleotides, comprising the sequences MJ3 (5′ AGT AGG ACC TAC ACC TGT CA 3′) (SEQ ID No: 5) and RT-EXT (5′ TTC CCA ATG CAT ATT GTG AG 3′) (SEQ ID No: 6), or composed of fragments of said sequences, or analogue sequences of said sequences comprising mutations of one or more nucleotides which do not essentially modify their ability to hybridise the region of the transcriptase gene comprising at least one mutation, followed by a second amplification step with a pair of primers, comprising the sequences: A35 (5′ TTG GTT GCA TAA ATT TTC CCA TTA GTC CTA TT 3′) (SEQ ID No: 7) and RT-IN (5′ TTC CCA ATG CAT ATT GTG AG 3′) (SEQ ID No: 8), or composed of fragments of said sequences, or analogue sequences of said sequences comprising mutations of one or more nucleotides which do not essentially modify their ability to hybridise the region of the reverse transcriptase gene comprising at least one mutation.

More preferentially, the amplification in step b according to the analytical method of the invention, with a pair of primers bordering a nucleic acid sequence liable to comprise at least one mutation in the gene coding for reverse transcriptase is performed with a pair of primers:

(SEQ ID No:5)

| | |
|---|---|
| MJ3 | (5′ AGT AGG ACC TAC ACC TGT CA 3′) and |

(SEQ ID No:6)

| | |
|---|---|
| RT-EXT | (5′ TTC CCA ATG CAT ATT GTG AG 3′), |

followed by a second amplification step with a pair of primers:

| | | |
|---|---|---|
| A35 | (5′ TTG GTT GCA TAA ATT TTC CCA TTA GTC CTA TT 3′) and | (SEQ ID No:7) |
| RT-IN | (5′ TTC CCA ATG CAT ATT GTG AG 3′) | (SEQ ID No:8) |

to obtain a DNA segment with 1530 base pairs ranging beyond codon 93 of the region coding for protease and beyond codon 503 of the region coding for polymerase (POL) and the vector from step c is a retroviral vector deleted from the region of the pol reading frame coding for HIV-1 reverse transcriptase ranging from the residues 2618 to 2872 inclusive, and comprising a single MluI restriction site.

The invention also relates to an analytical method to determine the susceptibility of an HIV virus to a reverse transcriptase inhibiting compound, consisting of adding said reverse transcriptase inhibiting compound or not, possibly at different concentrations, to the second cell host, before the infection of said host by the viral particles obtained in step e, and comprising in step g the comparison of the expression of the marker gene with and without reverse transcriptase inhibiting compound.

According to a third specific embodiment of the analytical method according to the invention, the PCR amplification in step b is carried out with a pair of primers bordering a nucleic acid sequence liable to comprise at least one mutation in the gene coding for integrase, and the vector in step c is a retroviral vector in which all or part of the gene coding for integrase is deleted.

Advantageously, the amplification in step b according to the analytical method of the invention, with a pair of primers bordering a nucleic acid sequence liable to comprise at least one mutation in the gene coding for integrase is performed with a pair of primers of a size between 10 and 50 oligonucleotides, comprising the sequences: INT B+-5′GTTACTAATAGAGGAAGACAAA3′(SEQ ID No: 9) and INT B− 5′TTTTGGTGTTATTAATGCT3′ (SEQ ID No: 10), or analogue sequences of said sequences comprising mutations of one or more nucleotides which do not essentially modify their ability to hybridise the region of the integrase gene comprising at least one mutation, followed by a second amplification step, with the pair of primers INT V+ 5′CACCCTAACTGACACAACAA3′ (SEQ ID No: 11) and INT V− 5′AAGGCCTTTCTTATAGCAGA3′ (SEQ ID No: 12), or analogue sequences of said sequences comprising mutations of one or more nucleotides which do not essentially modify their ability to hybridise the region of the integrase gene comprising at least one mutation.

More preferentially, the amplification in step b according to the analytical method of the invention, with a pair of primers bordering a nucleic acid sequence liable to comprise at least one mutation in the gene coding for integrase is performed with the pair of primers:

| | |
|---|---|
| INT B+ - | |
| 5′GTTACTAATAGAGGAAGACAAA3′ and | (SEQ ID No:9) |
| INT B− | |
| 5′TTTTGGTGTTATTAATGCT3′, | (SEQ ID No:10) |

followed by a second amplification step with the pair of primers:

| | | |
|---|---|---|
| INT V+ | 5′CACCCTAACTGACACAACAA3′ and | (SEQ ID No:11) |
| INT V− | 5′AAGGCCTTTCTTATAGCAGA3′, | (SEQ ID No:12) |

to obtain a DNA segment with 1460 base pairs ranging from residues 3950 to 5410 inclusive and the vector from step c is a retroviral vector deleted from the entire region of the pol reading frame coding for HIV-1 integrase ranging from the residues 4228 to 5093 inclusive and the region coding for the viral envelope between the positions 6343 and 7611 inclusive.

The invention also relates to an analytical method to determine the susceptibility of an HIV virus to an integrase inhibiting compound, consisting of adding said integrase inhibiting compound or not, possibly at different concentrations, during step e, before step f and comprising in step g the comparison of the expression of the marker gene with and without integrase inhibiting compound.

According to a fourth specific embodiment of the analytical method according to the invention, the PCR amplification in step b is carried out with a pair of primers bordering a nucleic acid sequence liable to comprise at least one mutation in the gene coding for envelope protein, and the vector from step c is a retroviral vector constructed from an HIV virus genome in which all or part of the gene coding for envelope protein is deleted.

Preferentially, the vector from step c is a retroviral vector deleted from the entire region coding for the extracellular portion of the gp41 sub-unit of the HIV-1 envelope, ranging from the residues 7745 to 8263 inclusive, the region of the HIV-1 genome forming the Rev response element (RRE).

Advantageously, the amplification in step b with a pair of primers bordering a nucleic acid sequence liable to comprise at least one mutation in the gene coding for envelope protein is performed with a pair of primers of a size between 10 and 50 oligonucleotides, comprising either the sequences: FIN-A: 5′TCAAATATTACAGGGCTGCT3′ (SEQ ID No: 13) and FIN-B: 5′TAGCTGAAGAGGCACAGG3′ (SEQ ID No: 14), either the sequences FuA: 5′AAGCAATGTATGCCCCTCCCAT3′ (SEQ ID No: 23) and FuB: 5′ GGTGGTAGCTGAAGAGGCACAGG3′ (SEQ ID No: 24) or composed of fragments of said sequences, or analogue sequences of said sequences comprising mutations of one or more nucleotides which do not essentially modify their ability to hybridise the region of the envelope gene comprising at least one mutation, followed by a second amplification step, with a pair of primers of a size between 10 and 50 oligonucleotides, comprising either the sequences: FIN-C: 5′CTATTAACAAGAGATGGTGG3′ (SEQ ID No: 15) and FIN-D: 5′TCCACCTTCTTCTTCGATT3′ (SEQ ID No: 16), or the sequences FuC: 5′ATATGAGGGACAATTGGAGAAGTGA3′ (SEQ ID No: 25), in combination with a mixture of the two following sequences: FuD1: 5′TCTGTCTCTCTCTCCACCTTCTTCTT3′ (SEQ ID No: 26) et FuD2: 5′TCTGTCTTGCTCTCCACCTTCTTCTT3′ (SEQ ID No: 27) or composed of fragments of said sequences, or analogue sequences of said sequences comprising mutations of one or more nucleotides which do not essentially modify their ability to hybridise the region of the envelope gene comprising at least one mutation.

More preferentially, the amplification in step b according to the analytical method of the invention, with a pair of primers bordering a nucleic acid sequence liable to comprise at least one mutation in the gene coding for envelope protein is performed with the pair of primers:

FIN-A: 5′TCAAATATTACAGGGCTGCT3′ (SEQ ID No: 13) and

FIN-B: 5′TAGCTGAAGAGGCACAGG3′ (SEQ ID No: 14) followed by a second amplification step, performed with the pair of primers:

| | | |
|---|---|---|
| FIN-C: | 5′CTATTAACAAGAGATGGTGG3′ and | (SEQ ID No:15) |
| FIN-D: | 5′TCCACCTTCTTCTTCGATT3′, | (SEQ ID No:16) |

to obtain a DNA segment with 965 base pairs ranging from the residues 7553 to 8517 inclusive and the vector in step c is a retroviral virus deleted from the entire region coding for the extracellular portion of the gp41 sub-unit of the HIV-1 envelope, ranging from the residues 7745 to 8263 inclusive, and comprises a single MulI restriction site.

More preferentially the analytical method of the invention allows amplification of a region of the envelope gene of any kind of viral HIV subtypes in particular A, B, C, D and E viral HIV subtypes by performing, the amplification in step b according to the analytical method of the invention, with a pair of primers bordering a nucleic acid sequence liable to comprise at least one mutation in the gene coding for envelope protein is performed with the pair of primers:

| | | |
|---|---|---|
| FuA: | 5′AAGCAATGTATGCCCCTCCCAT3′ and | (SEQ ID No:23) |
| FuB: | 5′GGTGGTAGCTGAAGAGGCACAGG3′ | (SEQ ID No:24) |

followed by a second amplification step, performed with the primer:

FuC: 5′ATATGAGGGACAATTGGAGAAGTGA3′ (SEQ ID No: 25) and a mixture of the following primers:

FuD1: 5′TCTGTCTCTCTCTCCACCTTCTTCTT3′ (SEQ ID No: 26)

FuD2: 5′TCTGTCTTGCTCTCCACCTTCTTCTT3′ (SEQ ID No: 27), said mixture being preferently carried out in a ratio comprised between (10%:90%) and (90%:10%) more preferently between (60%:40%) and (40%:60%),

to obtain a DNA segment with 805 base pairs ranging from the residues 7635 to 8440 inclusive and the vector in step c is a retroviral virus deleted from the entire region coding for the extracellular portion of the gp41 sub-unit of the HIV-1 envelope, ranging from the residues 7745 to 8263 inclusive, and comprises a single MulI restriction site.

Advantageously, the amplification in step b with a pair of primers bordering a nucleic acid sequence liable to comprise at least one mutation in the gene coding for envelope protein is preformed with a pair of primers of a size between 10 and 50 oligonucleotides, comprising the sequences: NEU-A: 5′TAGAAAGAGCAGAAGACAGTGGCAATG3′ (SEQ ID No: 17) and FIN-B: 5′TAGCTGAAGAGGCACAGG3′ (SEQ ID No: 14), or FuB: 5′ GGTGGTAGCTGAAGAGGCACAGG3′ (SEQ ID No: 24) or composed of fragments of said sequences, or analogue sequences of said sequences comprising mutations of one or more nucleotides which do not essentially modify their ability to hybridise the region of the envelope gene comprising at least one mutation, followed by a second amplification step, with a pair of primers of a size between 10 and 50 oligonucleotides, comprising the sequences: NEU-C: 5′GTGGGTCACAGTCTATTATGGGG3′ (SEQ ID No: 19) and FIN-D: 5′TCCACCTTCTTCTTCGATT3′ (SEQ ID No: 16), ) or a mixture of the following sequences: FuD1: 5′TCTGTCTCTCTCTCCACCTTCTTCTT3′ (SEQ ID No: 26) et FuD2: 5′TCTGTCTTGCTCTCCACCTTCTTCTT3′ (SEQ ID No: 27), or composed of fragments of said sequences, or analogue sequences of said sequences comprising mutations of one or more nucleotides which do not essentially modify their ability to hybridise the region of the envelope gene comprising at least one mutation.

More preferentially, the amplification in step b according to the analytical method of the invention, with a pair of primers bordering a nucleic acid sequence liable to comprise at least one mutation in the gene coding for envelope protein is performed with the pair of primers:

NEU-A: 5′TAGAAAGAGCAGAAGACAGTGGCAATG3′ (SEQ ID No: 17) and

FIN-B: 5′TAGCTGAAGAGGCACAGG3′ (SEQ ID No: 14) followed by a second amplification step, performed with the pair of primers:

| | |
|---|---|
| NEU-C: | |
| 5′GTGGGTCACAGTCTATTATGGGG3′ and | (SEQ ID No:19) |
| FIN-D: | |
| 5′TCCACCTTCTTCTTCGATT3′, | (SEQ ID No:16) |

to obtain a DNA segment with 2320 base pairs ranging from the residues 6197 to 8517 inclusive and the vector in step c is a retroviral vector deleted from the entire region coding for the majority of the gp120 sub-unit and the extracellular portion of the gp41 sub-unit of the HIV-1 envelope, ranging from the residues 6480 to 8263 inclusive, and comprises a single MulI restriction site.

More preferentially, the amplification in step b according to the analytical method of the invention, with a pair of primers bordering a nucleic acid sequence liable to comprise at least one mutation in the gene coding for envelope protein is performed with the pair of primers:

| | |
|---|---|
| NEU-A: | |
| 5′TAGAAAGAGCAGAAGACAGTGGCAATG3′ and | (SEQ ID No:17) |
| FuB: | |
| 5′GGTGGTAGCTGAAGAGGCACAGG3′, | (SEQ ID No:24) |

followed by a second amplification step, performed with the pair of primers:

NEU-C: 5′GTGGGTCACAGTCTATTATGGGG3′ (SEQ ID No: 19) and a mixture of the following primers

FuD1: 5′TCTGTCTCTCTCTCCACCTTCTTCTT3′ (SEQ ID No: 26) and

FuD2: 5′TCTGTCTTGCTCTCCACCTTCTTCTT3′ (SEQ ID No: 27), said mixture being preferently carried out in a ratio comprised between (10%:90%) and (90%:10%) more preferently between (60%:40%) and (40%:60%),

to obtain a DNA segment with 2118 base pairs ranging from the residues 6322 to 8440 inclusive and the vector in step c is a retroviral vector deleted from the entire region coding for the majority of the gp120 sub-unit and the extracellular portion of the gp41 sub-unit of the HIV-1 envelope, ranging from the residues 6480 to 8263 inclusive, and comprises a single MulI restriction site.

Advantageously, the amplification in step b with a pair of primers bordering a nucleic acid sequence liable to comprise at least one mutation in the gene coding for envelope protein is performed with a pair of primers of a size between 10 and 50 oligonucleotides, comprising the sequences: E00: 5′TAGAAAGAGCAGAAGACAGTGGCAATGA3′ (SEQ ID No: 19) and ES8B: 5′CACTTCTCCAATTGTCCCTCA3′ (SEQ ID No: 20), or composed of fragments of said sequences, or analogue sequences of said sequences comprising mutations of one or more nucleotides which do not essentially modify their ability to hybridise the region of the envelope gene comprising at least one mutation, followed by a second amplification step, with a pair of primers of a size between 10 and 50 oligonucleotides, comprising the sequences: E20: 5′GCGCCACACATGCCTGTGTACCCACAG3′ (SEQ ID No: 21) and E115: 5′AGAAAAATTCCCCTCCACAATTAA3′ (SEQ ID No: 22), or analogue sequences of said sequences comprising mutations of one or more nucleotides which do not essentially modify their ability to hybridise the region of the protease gene comprising at least one mutation.

More preferentially, the amplification in step b according to the analytical method of the invention, with a pair of primers bordering a nucleic acid sequence liable to comprise at least one mutation in the gene coding for envelope protein is performed with the pair of primers:

| | |
|---|---|
| E00: | |
| 5′TAGAAAGAGCAGAAGACAGTGGCAATGA3′ and | (SEQ ID No:19) |
| ES8B: | |
| 5′CACTTCTCCAATTGTCCCTCA3′, | (SEQ ID No:20) |

followed by a second amplification step, performed with the pair of primers:

| | |
|---|---|
| E20: | |
| 5′GGGCCACACATGCCTGTGTACCCACAG3′ and | (SEQ ID No:21) |
| E115: | |
| 5′AGAAAAATTCCCCTCCACAATTAA3′, | (SEQ ID No:22) |

to obtain a DNA segment with 938 base pairs ranging from the residues 6426 to 7364 inclusive and the vector in step c is a retroviral vector deleted from the region, coding for the domains ranging from the loop V1 to the loop V3 of the HIV-1 envelope ranging from 6617 to 7250 inclusive and comprises a single NheI restriction site.

The invention also relates to an analytical method to determine the susceptibility of an HIV virus to a fusion inhibiting compound targeting HIV-1 gp41 protein, consisting of performing the amplification in step b either with the pair of primers SEQ ID NO: 13, SEQ ID No: 14 followed by a second amplification with the pair of primers SEQ ID No: 15, SEQ ID No: 16, or with the pair of primers SEQ ID No: 17, SEQ ID No: 18 followed by a second amplification with the pair of primers SEQ ID No: 18, SEQ ID No: 16, adding said fusion inhibiting compound or not, possibly at different concentrations, during the culture of the cell host obtained in step e, before step f and comprising in step g the comparison of the expression of the marker gene with and without fusion inhibiting compound targeting HIV-1 gp41.

The invention also relates to an analytical method to determine the susceptibility of an HIV virus to a compound inhibiting the entry of said HIV virus into a target cell, consisting of performing the amplification in step b with the pair of primers SEQ ID No: 17 and SEQ ID No: 18 followed by a second amplification with the pair of primers SEQ ID No: 18 and SEQ ID No: 16, adding said entry inhibiting compound or not, possibly at different concentrations, to the cell host obtained in step e before the infection in step f and comprising in step g the comparison of the expression of the marker gene with and without entry inhibiting compound.

The invention also relates to an analytical method to determine the susceptibility of an HIV virus to the inhibitory action of antibodies, consisting of performing the amplification in step b with the pair of primers SEQ ID No: 17 and SEQ ID No: 18 followed by a second amplification with the pair of primers SEQ ID No: 18 and SEQ ID No: 16, adding said the antibodies during the culture step e, and comprising in step g the comparison of the expression of the marker gene with and without antibodies.

The invention also relates to an analytical method to determine the tropism of an HIV virus for a cell receptor, consisting of performing the amplification in step b with the pair of primers SEQ ID No: 17 and SEQ ID No: 18 followed by a second amplification with the pair of primers SEQ ID No: 18 and SEQ ID No: 16, performing the infection in step f with the viral particles obtained in step e on two separate cell hosts and comprising in step g the comparison of the expression of the marker gene by each of the two separate cell hosts.

Advantageously, the cell hosts used for infection in step f according to the analytical method of the invention are selected from cell hosts expressing the CCR5 receptor or the CXCR4 receptor.

The invention also relates to an analytical method to determine the susceptibility of an HIV virus to an inhibiting compound targeting HIV-1 co-receptors, consisting of performing the amplification in step b with the pair of primers SEQ ID No: 17 and SEQ ID No: 18 followed by a second amplification with the pair of primers SEQ ID No: 18 and SEQ ID No: 16, adding said inhibiting compound targeting HIV-1 co-receptors or not, possibly at different concentrations, during the culture step e, the infection in step f being performed on two separate cell hosts and comprising in step g the comparison of the expression of the marker gene by each of the two separate cell hosts.

The invention also relates to an analytical method to determine the tropism of an HIV virus for a cell receptor, consisting of performing the amplification in step b with the pair of primers SEQ ID No: 19 and SEQ ID No: 20 followed by a second amplification with the pair of primers SEQ ID No: 21 and SEQ ID No: 22, infecting in step f two separate cell hosts with the viral particles obtained in step e and comprising in step g a comparison of the expression of the marker gene by each of the two separate cell hosts.

The invention also relates to an analytical method to determine the susceptibility of an HIV virus to an inhibiting compound targeting HIV-1 co-receptors, consisting of performing the amplification in step b with the pair of primers SEQ ID No: 19 and SEQ ID No: 20 followed by a second amplification with the pair of primers SEQ ID No: 21 and SEQ ID No: 22, adding said inhibiting compound targeting HIV-1 co-receptors or not, possibly at different concentrations, during the culture in step d, performing the infection in step f with the viral particles obtained in step e on two separate cell hosts and comparing in step g the expression of the marker gene by each of the two separate cell hosts.

The invention also relates to an analytical method to determine the infectivity or replicative capacity of an HIV virus consisting of comparing in step g the expression of the marker gene by the second cell host infected with the viral particles obtained by applying steps a to f to a biological specimen from a patient, and the expression of the marker gene by the same second cell host infected with the reference viral particles obtained by applying steps a to f to a specimen containing a reference virus.

Advantageously, the reference viral particles from a reference virus are viral particles obtained by applying steps a to f to a biological specimen from the same patient at an earlier stage of treatment or before said treatment.

The invention also relates to an analytical method to determine the virulence of an HIV virus consisting of performing the amplification in step b either with the pair of primers SEQ ID No: 13, SEQ ID No: 14 followed by a second amplification with the pair of primers SEQ ID NO: 15 SEQ ID No: 16, or with the pair of primers SEQ ID No: 17 SEQ ID No: 18 followed by a second amplification with the pair of primers SEQ ID No: 18, SEQ ID No: 16, and measuring, during the infection in step f, the cytopathogenic effect produced on the second cell host.

Advantageously, the cytopathogenic effect produced during the infection in step f on the second cell host is measured by means of cytotoxicity techniques such as measuring the syncytial induction, apoptosis induction or using flow cytometry.

The invention also relates to an analytical method to determine the susceptibility of an HIV virus to hydroxyurea, consisting of adding hydroxyurea or not, possibly at different concentrations, during the culture step e, and performing in step g the comparison of the expression of the marker gene with and without hydroxyurea.

Preferentially, the duration of the culture step e is between 12 hours and 72 hours, more preferentially between 24 hours and 48 hours.

The invention also relates to a kit for implementing a method to analyse a phenotypic characteristic of HIV viruses present in a biological specimen from a patient characterised in that it comprises:

i) a pair of primers bordering a nucleic acid sequence of the viral genome liable to comprise at least one mutation,

ii) a vector comprising the parts of an HIV virus genome required for viral replication except for the segment amplified with the primers defined in i and the gene coding for the envelope protein,

iii) a second vector comprising a gene coding for envelope protein,

iv) a first cell host liable to be infected by an HIV virus,

v) a second cell host liable to be infected by an HIV virus and comprising a marker gene that can only be activated following viral infection,

vi) the products and reagents required to carry out PCR amplification,

vii) the products and reagents used to detect the expressed marker.

Advantageously, the kit according to the invention comprises:

i) the sequence primer pairs:

SEQ ID No:1 and SEQ ID No:2
SEQ ID No:3 and SEQ ID No:4

ii) a retroviral vector deleted from the region of the pol reading frame coding for HIV-1 protease ranging from the residues 1505 to 2565 inclusive, deleted from the envelope region and comprising a single MluI restriction site,

viii) a pseudotype virus with a gene coding for an envelope protein,

iv) a first cell host liable to be infected by an HIV virus,

v) a second cell host liable to be infected by an HIV virus and comprising a marker gene that can only be activated following viral infection,

vi) the products and reagents required to carry out PCR amplification,

vii) the products and reagents used to detect the expressed marker.

Advantageously, the kit according to the invention comprises:

i) the sequence primer pairs:

SEQ ID No:5 and SEQ ID No:7
SEQ ID No:6 and SEQ ID No:8

ii) a retroviral vector deleted from the region of the pol reading frame coding for HIV-1 reverse transcriptase ranging from the residues 2618 to 2872 inclusive, and comprising a single MluI restriction site,

iii) a pseudotype virus with a gene coding for an envelope protein,

iv) a first cell host liable to be infected by an HIV virus,

v) a second cell host liable to be infected by an HIV virus and comprising a marker gene that can only be activated following viral infection,

vi) the products and reagents required to carry out PCR amplification,

vii) the products and reagents used to detect the expressed marker.

Advantageously, the kit according to the invention comprises:

i) the sequence primer pairs:

SEQ ID No:9 and SEQ ID No:10
SEQ ID No:11 and SEQ ID No:12

ii) a retroviral vector deleted from the region of the pol reading frame coding for HIV-1 integrase ranging from the residues 4228 to 5093 inclusive and the region coding for the viral envelope between the positions 6343 and 7611 inclusive,

iii) a pseudotype virus with a gene coding for an envelope protein,

iv) a first cell host liable to be infected by an HIV virus,

v) a second cell host liable to be infected by an HIV virus and comprising a marker gene that can only be activated following viral infection,

vi) the products and reagents required to carry out PCR amplification,

vii) the products and reagents used to detect the expressed marker.

Advantageously, the kit according to the invention comprises:

i) the sequence primer pairs:

SEQ ID No:13 and SEQ ID No:14
SEQ ID No:15 and SEQ ID No:16

ii) a retroviral vector deleted from the entire region coding for the extracellular portion of the HIV-1 envelope gp41 sub-unit, ranging from the residues 7745 to 8263 inclusive, and comprising a single MulI restriction site,

iv) a first cell host liable to be infected by an HIV virus,

v) a second cell host liable to be infected by an HIV virus and comprising a marker gene that can only be activated following viral infection,

vi) the products and reagents required to carry out PCR amplification,

vii) the products and reagents used to detect the expressed marker.

Advantageously, the kit according to the invention comprises:

i) the sequence primer pairs:

SEQ ID No:17 and SEQ ID No:14
SEQ ID No:18 and SEQ ID No:16

ii) a retroviral vector deleted from the entire region coding for the majority of the gp120 sub-unit and the extracellular portion of the HIV-1 envelope gp41 sub-unit, ranging from the residues 6480 to 8263 inclusive, and comprising a single MulI restriction site,

iv) a first cell host liable to be infected by an HIV virus,

v) a second cell host liable to be infected by an HIV virus and comprising a marker gene that can only be activated by viral particles,

vi) the products and reagents required to carry out PCR amplification,

vii) the products and reagents used to detect the expressed marker.

Advantageously, the kit according to the invention comprises:

i) the sequence primer pairs:

SEQ ID No:19 and SEQ ID No:20
SEQ ID No:21 and SEQ ID No:22

ii) a retroviral vector deleted from the region, coding for the domains ranging from the loop V1 to the loop V3 of the HIV-1 envelope, ranging from 6617 to 7250 inclusive, and comprising a single NheI restriction site,

iv) a first cell host liable to be infected by an HIV virus,

v) a second cell host liable to be infected by an HIV virus and comprising a marker gene that can only be activated following viral infection,

vi) the products and reagents required to carry out PCR amplification,

vii) the products and reagents used to detect the expressed marker.

The invention's other advantages and characteristics are illustrated in the following examples referring to the following figures:

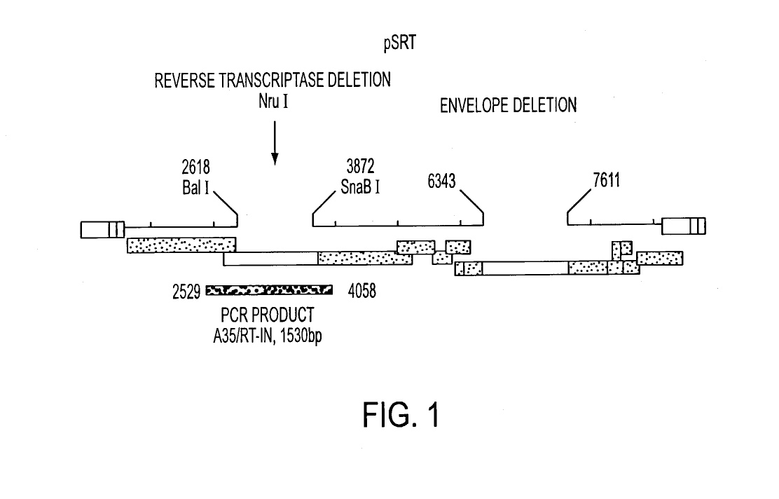

FIG. 1 is a schematic representation of pSRT plasmid. The region coding for pNL4-3xcenv reverse transcriptase is deleted by means of BalI-SnaBI digestion. The resulting linearisation of pSRT is carried out using Nru I.

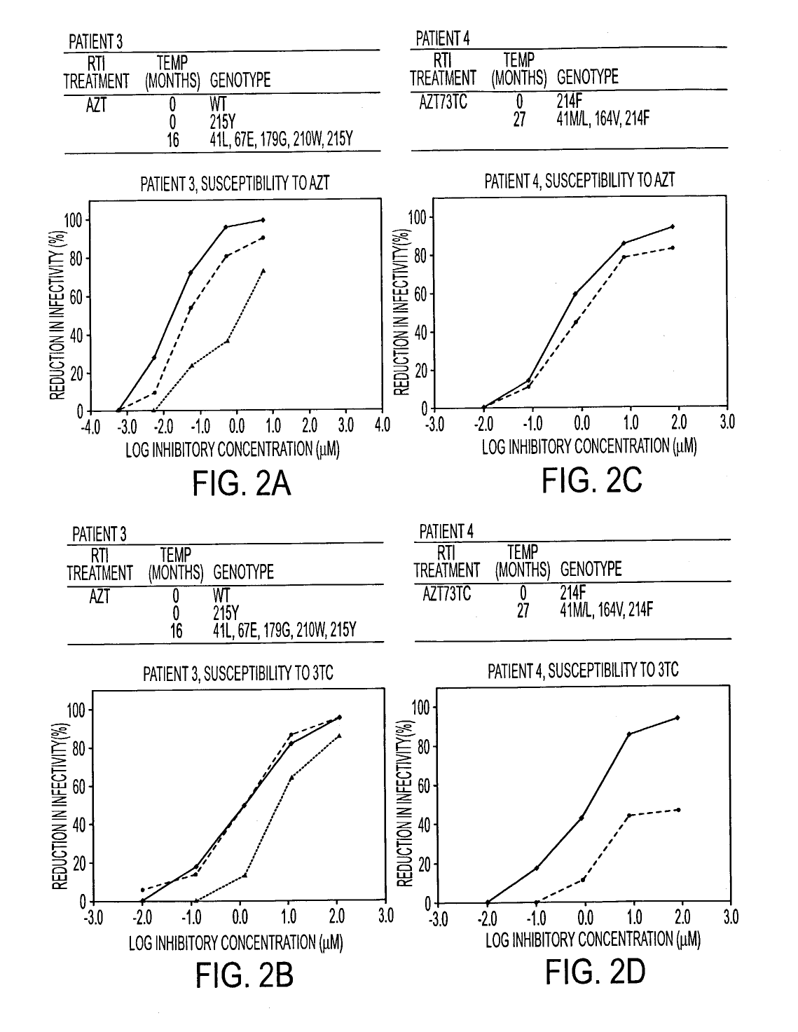

FIG. 2 illustrates the dose response effect curves obtained for two patients versus AZT and 3TC, before and after treatment with reverse transcriptase inhibitors.

The details of the treatment, sampling intervals and genotypes are given in the figure headings.

The curves show the inhibition of the infection by a recombinant virus of P4 cells treated either with zidovudine (AZT, panels A and C) or lamivudine (3TC, panels B and D), according to the technique described below in the material and methods section. For patient 1, the specimens were tested at 0 ♦, 9 ● and 18 □ months after treatment, and for patient 2 at 0 ♦ and 27 ● months after treatment.

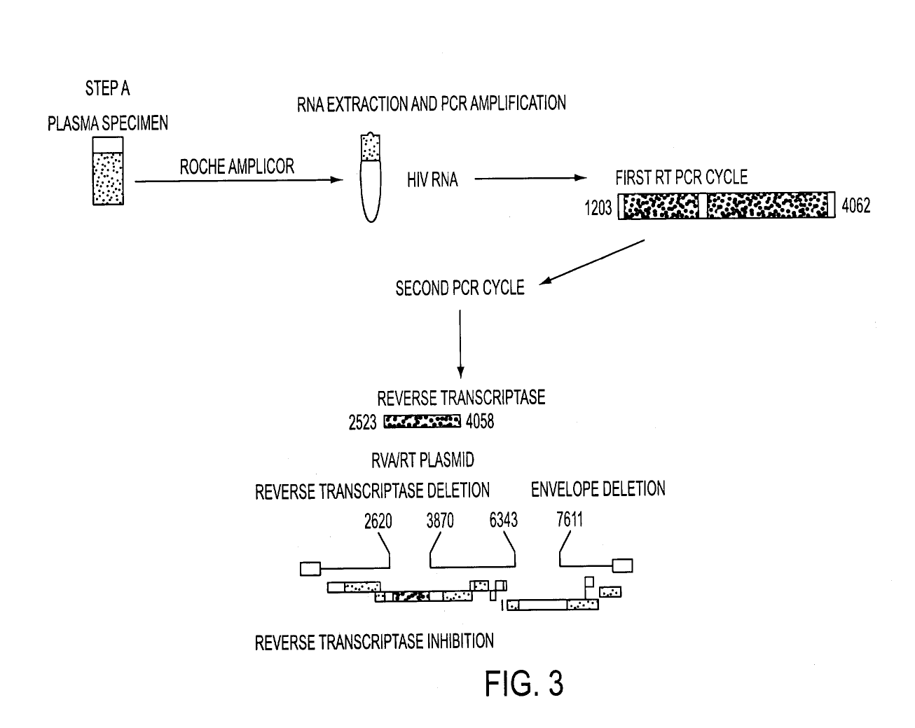

FIG. 3 is a diagram of the first steps a and b of a specific embodiment of the method according to the invention. The diagram illustrates the extraction step a and amplification step b on the reverse transcriptase sequences extracted from plasma of a patient using RT PCR of the method according to the invention and the construction diagrams of the pRVA/RT plasmid used subsequently in step d.

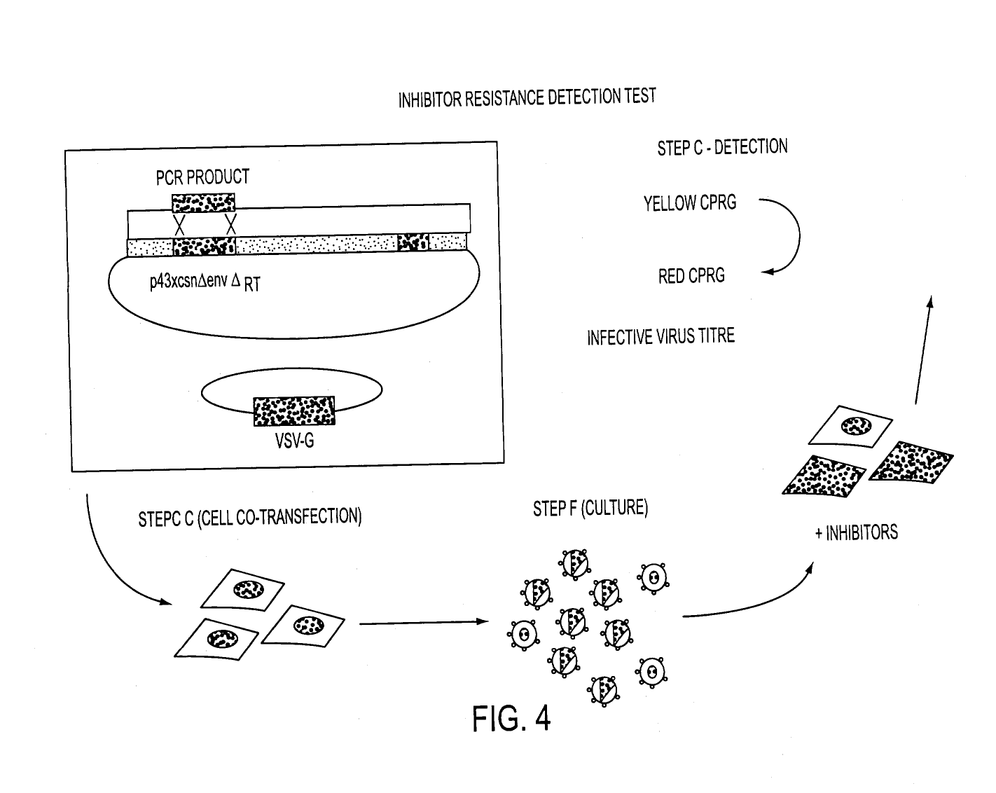

FIG. 4 is a diagram of step c to g of a specific embodiment of the method according to the invention. This diagram illustrates the co-transfection step d in HeLa/293T cells of nucleic acids amplified in step b, of a first RT p43xcsnΔenv plasmid constructed from an HIV virus genome, not comprising a nucleic acid segment corresponding to all or part of the nucleic acid sequence amplified in step b or a fragment of the nucleic acid sequence coding for envelope protein and a second pVSV-G plasmid comprising the sequence coding for envelope protein; the culture step e being used to produce viral particles, step f consisting of transfecting the viral particles obtained in step e in P4 cells, previously incubated in the presence of serial dilutions of different reverse transcriptase inhibitors or not, said P4 indicator cells comprising a system to express the gene coding for beta-galactosidase enzyme that can only be activated by tat activation sequences expressed by the recombinant virus, and step g consisting of detecting and/or quantifying the beta-galactosidase by means of CPRG substrate.

I—Material and Methods

I.1—Polymerisation Chain Reaction (PCR) Amplification

The RNA is isolated from the plasma of patients by means of a Roche Amplicor® kit (Roche Diagnostics, 38242 Meylan Cedex, France), and the genes of interest are isolated by means of a reverse transcriptase and a subsequent PCR reaction.

The amplification of the region coding for reverse transcriptase (RT) is performed by means of external primers MJ3 (5′ AGT AGG ACC TAC ACC TGT CA 3′) (SEQ ID No: 5, appended) and RT-EXT (5′ TTC CCA ATG CAT ATT GTG AG 3′) (SEQ ID No: 6, appended) and internal primers A35 (5′ TTG GTT GCA TAA ATT TTC CCA TTA GTC CTA TT 3′) (SEQ ID No: 7, appended) and RT-IN (5′ TTC CCA ATG CAT ATT GTG AG 3′) (SEQ ID No: 8, appended) with an initial cycle at 50° C. (30 minutes) and at 94° C. (2 minutes), followed by 40 cycles at 94° C. (30 seconds), 55° C. (30 seconds) and 68° C. (90 seconds) and a final extension step at 98° C. for 10 minutes. This amplifies a product of 1530 bp which ranges beyond codon 93 of the region coding for protease and beyond codon 503 of the region coding for polymerase (pol).

The products obtained by PCR are purified on QuiaAmp® columns and analysed in terms of their size, degree of purity and approximate concentration by electrophoresis on agar.

I.2—Genotyping

The nucleotide sequences of the regions coding for reverse transcriptase are determined by automatic sequencing of the dideoxinucleotide chain terminal of the unprocessed PCR products.

I.3—Plasmids

The HIV-1 molecular clones used in the analytical method are derived from pNL4-3.

The reverse transcriptase-deleted plasmid is constructed by the modifying the mutated pNL4-3xcsnΔenv to comprise single restriction sites, SnaBi in position 3872 and NruI in position 3892.

The enzymes Ba I and SnaB I are used to remove the region coding for reverse transcriptase (between positions 2618 and 2872) and the linearisation of the resulting pSRT plasmid is performed by means of the Nru I enzyme. The VSV-G envelope glycoprotein is expressed in the transfected cells by the pVSV plasmid which contains the vsv-g coding sequence under the control of a CMV promoter.

I.4—Cell Cultures.

HeLa, 293 T and P4 cells are cultured in DMEM medium supplemented with 10% foetal calf serum (FCS), 50 IU/ml of penicillin and 50 μg/ml of streptomycin. The P4 cells are HelA-CD4, LTR-LacZ cells wherein the expression of beta-galactosidase can only be induced by a HIV transactivating Tat protein, enabling as a result a precise quantification of the infectivity or replicative capacity of HIV-I viruses based on a single replication cycle (Charneau, P., Mirambeau, G., Roux, P., Paulous, S., Buc, H. and Clavel, F. (1994) “HIV-1 reverse transcription. A termination step at the center of the genome”. J Mol Biol 241(5), 651-652). The P4 cells are cultured in the presence of 500 μg/ml of geneticine.

II.—Determination of the Susceptibility of an HIV Virus to Reverse Transcription Inhibitor (RTI).

The determination of the susceptibility of an HIV virus is performed as follows: 293 T cells are transfected with 7.5 μg of pSRT plasmid linearised with NruI, 0.1 μg of pVSV-G plasmid and 0.5 and 1 μg of product from the HIV reverse transcriptase PCR reaction. The transfection precipitate is removed from the cells after 18 hours of incubation and the fresh growth medium is added. After 24 hours of culture, the supernatant is clarified by centrifugation (500 g, 15 minutes) and transferred on P4 indicator cells which have been pre-incubated with serial dilutions of a reverse transcriptase inhibitor, in triplicate wells, for four hours. The range of inhibitor concentrations used varies according to the compounds. The signal produced by activating the marker gene was developed with CPRG for 48 hours, as for the analysis of the susceptibility to a reverse transcriptase inhibitor and the IC₅₀ index was calculated using the median effect equation.

III—Optimisation of the Analytical Method to Determine the Susceptibility of an HIV Virus to Reverse Transcriptase Inhibitors.

The pSRT plasmid comprising a deletion in the region coding for pol ranging from reverse transcriptase codon 24 (base 2618) to reverse transcriptase codon 432 (base 3872) includes all the mutations associated with a resistance phenomenon known to date. The homologous sequences of the PCR reverse transcriptase product ranging 88 base pairs upstream and 186 base pairs downstream from the deletion in pSRT. The transfections to determine the susceptibility to reverse transcriptase inhibitors are performed with a 293T cell line with a strong capacity to be transfected rather than with HeLa cells. This is not a problem since the cells are eliminated from the supernatant containing the virus by centrifugation before the transfer of P4 cells.

The transfer conditions are optimised by means of a checkerboard test. The variation of the plasmid/PCR product ratio does not modify the quantity of p24 or reverse transcriptase produced or the reaction rate with CPRG significantly. Given that the circular plasmid, pVSV-G, seems to be extremely toxic for 293T cells, the quantity of said plasmid was reduced from 3 μg to 0.1 μg in the transfection mixture, resulting in high p24 yields (>20 ng/ml compared to 9.8 ng/ml).

Given that the early phases of virus replication, including reverse transcription, take place in indicator P4 cells in this type of determination, these cells are treated with serial dilutions of reverse transcriptase inhibitors.

The inhibitor concentration ranges are selected as a function of the cellular toxicity of each compound and the IC₅₀/IC₉₀ ratio for the susceptibility of resistant isolates (Table 1). For example, since the IC₅₀ index for abacavir for P4 cells is approximately 250 μM while the IC₅₀ index for this compound for the native strain of the virus is approximately 3 μM, the detection of the resistance is limited by the toxicity. A range of four dilutions in series, starting at 200 μM, was used for abacavir, enabling the detection of up to 60 times more resistance.

In addition, since the toxicity of AZT for P4 cells is high (>300 μM) while the IC₅₀ index is considerably lower, a wider range of dilutions was used (1/10 serial dilution from 5 μM) so as to enable the detection of high levels of resistance (up to 100 times).

For RVA reverse transcriptase, the IC₅₀ index is used rather than the IC₉₀ index since the detection of the IC₉₀ index for resistant viruses could require toxic levels of compound for most reverse transcriptase inhibitors.

TABLE I
Susceptibility of NL4-3 reference virus to reverse transcriptase inhibitors (RTI).

| | | | | Geometric | | |
|---|---|---|---|---|---|---|
| | Drug concentrations | | Dilution | mean IC₅₀^{a} | Standard | Maximum detectable |
| RT inhibitors | used | | steps | (μM) | deviation^{a} | susceptibility^{b} |
| AZT | 50 μm-5 | nM | 10× | 0.018 | 2.7 | 2700 |
| 3TC | 200-0.02 | μM | 10× | 1.512 | 2.2 | 130 |
| C4T | 100-0.01 | μM | 10× | 0.444 | 2.7 | 220 |
| DDI | 100-0.01 | μM | 10× | 1.613 | 2.5 | 60 |
| Abacavir | 200-0.8 | μM | 4× | 2.229 | 1.8 | 90 |
| Effavirenz | 100-0.16 | nM | 5× | 0.716 | 2.2 | 140 |
| Nevirapine | 50 μM-5 | nM | 10× | 0.037 | 2.0 | 1300 |

^{a}geometric mean and standard deviation for 20 repeated tests for reverse transcriptase inhibitors.
^{b}maximum approximate difference for the IC₅₀ and IC₉₀ indices between NL43 and the virus under test which may be measured using the drug concentration range given

The analytical method to determine the susceptibility of HIV virus to reverse transcriptase inhibitors gives a Standard Deviation of the geometric mean for 20 tests between 1.78 (abacavir) and 2.7 (D4T and AZT). The median standard deviation for the reverse transcriptase inhibitors (RTI) tested (AZT, 3TC, D4T, DDI, abacavir, effavirenz and nevirapine) is 2.2. So as to simplify the automatic interpretation of the results of the patient specimens, an arbitrary Resistance Index (RI) value, RI=5, was defined as the minimum reduction of the susceptibility to an RTI considered as being significantly reduced with reference to NL43.

To determine whether NL43 is a suitable reference virus for comparison with clinical isolates, a panel of specimens taken from patients with a standard treatment were tested on 3 RVA replicates for their susceptibility to reverse transcriptase inhibitors.

The median IC₅₀ for 22 viruses tested tends to be slightly higher than that found for NL43 virus with a median RI of approximately 0.92 (for stavudine) and 1.22 (lamivudine). Although the RI is less than the defined limit of 5 out of the total inhibitors for most of the specimens tested, the RI range appears to be wide, particularly for non-nucleoside inhibitors. In particular, a resistance index of 11.0 for nevirapine was obtained for a virus, and this virus comprised a mutation on codon 98 (A for S) of the reverse transcriptase, which had previously been involved in NNRTI resistance.

IV—Reproducibility

The reproducibility of the method to determine the susceptibility of recombinant virus (RVA) to reverse transcriptase inhibitors (RTI) was evaluated with a series of tests on 5 specimens, selected so as to represent a wide range of susceptibility profiles, between 4 and 8 tests per specimen, using RNA preparations and separate PCR reactions.

The inter-test variation for the determination of the susceptibility of HIV viruses to reverse transcriptase inhibitors indicates that in some cases, there is a difference greater than 5 between the maximum RI and the minimum RI found for repeated determinations (Table 2); however, the standard deviation of the geometric mean is maintained equal to 2.2 in all cases except one (R4, AZT). In three of the specimens, the RI obtained during the repeated tests varies between <5 and >5 for compounds for which the viruses may be moderately resistant (RI not greater than 12).

TABLE 2
Reproducibility of RTI susceptibility.

| Specimen | | | | | | | | | |
|---|---|---|---|---|---|---|---|---|---|
| Number of | | Resistance Index^{b} | | | | | | | |
| tests) | Genotype^{a} | | AZT | 3TC | D4T | DDI | Abacavir | Efavirenz | Nevirapine |
| R1 | 69N/T, 70R | GM^{c} | 1.5 | 1.3 | 1.4 | 1.4 | 1.1 | 1.0 | 2.2 |
| (6) | | SD^{d} | 1.5 | 1.5 | 1.4 | 1.6 | 1.2 | 1.1 | 1.8 |
| | | max/min^{e} | 2.9 | 2.8 | 2.2 | 2.8 | 1.6 | 1.2 | 3.8 |
| | | n > 4/N^{f} | 0/6 | 0/6 | 0/6 | 0/6 | 0/6 | 0/6 | 0/6 |
| R2 | 41L, 62V, 67N, 69N, 751, | GM^{c} | 1232.6 | >f | 43.5 | 26.9 | > | 74.9 | > |
| (6) | 77L, 115F, 116Y, 151M, | SD^{d} | 1.6 | na | 2.1 | 1.7 | na | 1.7 | na |
| | 181C, 184V, 190A, 208Y, | max/min^{e} | 3.8 | na | 8.1 | 4.1 | na | 3.9 | na |
| | 215F, 219Q | n > 4/N^{f} | 6/6 | 6/6 | 6/6 | 6/6 | 6/6 | 6/6 | 6/6 |
| R3 | 184V, 21SF | GM^{c} | 13.2 | > | 3.2 | 3.1 | 4.3 | 1.0 | 1.2 |
| (6) | | SD^{d} | 2.7 | na | 1.8 | 1.6 | 1.4 | 1.0 | 1.3 |
| | | max/min^{e} | 8.9 | na | 5.7 | 3.3 | 2.4 | 1.0 | 1.6 |
| | | n > 4/N^{f} | 5/6 | 1/6 | 1/6 | 1/6 | 4/6 | 0/6 | 0/6 |
| R4 | 41L, 67N, 69D, 184V, | GM^{c} | 137.5 | > | 3.6 | 3.1 | 4.3 | 1.0 | 1.2 |
| (6) | 190A, H208Y, 210W, 215Y | SD^{d} | 1.8 | na | 2.0 | 1.8 | 1.6 | 1.6 | na |
| | | max/min^{e} | 4.5 | na | 4.1 | 1.7 | 3.3 | 3.9 | na |
| | | n > 4/N^{f} | 5/5 | 5/5 | 3/5 | 5/5 | 3/5 | 5/5 | 5/5 |
| R5 | 215Y, 41L, 74V, 100I, 103N | GM^{c} | 3.3 | 1.8 | 1.7 | 2.6 | 1.7 | > | 580.5 |
| (6) | SD^{d} | 1.6 | 1.7 | 1.8 | 1.6 | 1.6 | na | 1.8 | |
| | max/min^{e} | 3.0 | 3.7 | 3.7 | 2.5 | 2.9 | na | 2.2 | |
| | n > 4/N^{f} | 1/5 | 0/5 | 0/5 | 0/5 | 0/5 | 5/5 | 5/5 | |

(a) Only the amino acid substitutions already known to be associated with resistance to reverse transcriptase inhibitors are indicated.

(b) The Resistance Index is the ratio of the IC₅₀ in the specimen with reference to that of NL43 determined in parallel.

(c) Geometric mean.

(d) Standard deviation of geometric mean.

(e) The highest resistance index obtained in the tests divided by the lowest.

(f) The number of tests giving a resistance index >4 classifying the virus as resistant in the total number of tests conducted.

(g) An IC₅₀ above the detectable range in all the tests is indicated by >. In these cases, a standard deviation and a minimum/maximum are not applicable (na).

The phenotypic results obtained for these reverse transcriptase inhibitors demonstrate consistency with the genotypic profiles of the specimens.

Specimen R2, which shows a high degree of resistance with reference to all the compounds tested, comprises multiple mutations including those of the multi-compound resistance complex (62V, 75I, 77L, 116Y and 151M) which induces resistance to RTI nucleotides, 3TC resistance associated with the 184V mutation and mutations known to induce reduced NNRTI susceptibility (181C, 190A).

Specimen R3 shows a high level of resistance to 3TC, again modulated by the 184V mutation, with a considerable variation of the RI for AZT which probably reflects the inconsistency of the elimination of resistance induced by 215F by 184V.

In specimen R4, such an elimination is counteracted by the presence of multiple mutations including a 208Y mutation.

The R5 specimen remains susceptible to AZT despite a 41L mutation and a 215Y mutation due to the effects of the 100I mutation which, in combination with 103N, is responsible for the high levels of resistance observed with respect to efavirenz and nevirapine.

V—Validation of the Analytical Method for the Determination of the Susceptibility of HIV Viruses to Fusion Inhibitors.

The recombinations obtained by performing the amplification of the step (b) with the pair of primers FuA: 5′AAGCAATGTATGCCCCTCCCAT3′ (SEQ ID No: 23) et FuB: 5′ GGTGGTAGCTGAAGAGGCACAGG3′ (SEQ ID No: 24) followed by a second amplification step, performed with the primer FuC: 5′ATATGAGGGACAATTGGAGAAGTGA3′ (SEQ ID No: 25) and a mixture of the following primers: FuD1: 5′TCTGTCTCTCTCTCCACCTTCTTCTT3′ (SEQ ID No: 26) FuD2: 5′TCTGTCTTGCTCTCCACCTTCTTCTT3′ (SEQ ID No: 27) are highly efficient.

That is, in order to obtain a satisfactory viral production 10-50 ng from the PCR product obtained, are generally enough to carry out the recombination step. In the analytical method to determine the fusion phenotypical feature in which the amplification step (b) is performed with the above primers, a viral production corresponding to 50-400 ng of p24 protein is obtained after transfection. The increase on the optical density obtained after the infection of cells is linear if the infection is performed under these conditions.

In order to validate the analytical mehod to determine the susceptibility the of HIV viruses to fusion inhibitors, two different fusion inhibitors has been used: a peptide derived from the sequence of the distal helix in HIV-gp41 (named DP178 or T20) and a betulinic acid derivative (RPR103611). For each inhibitor the decrease in sensitivity from one or more resistant viruses (prior identified as resistants by other methods), by comparison to two reference viruses: a primary plasma virus (T5A1) and a culture adapted virus (LAI) has been performed. Alle viruses has been produced by recombination.

Results

V.1—DP178 or T20 Inhibitor.

The IC50 index for a highly resistant virus NL-DIM ( Rimski L. T. et al. J. Virol. 1998, vol 72, pages 986-993) and for the NL4.3 virus (which is partially resistant) has been measured by reference to the DP178 inhibitor.

The DIM highly resistant virus presents an increase of its IC50 index of more than 80 times by reference to T5Al virus and more than 100 times by reference to LAI virus.

The partially resistant virus NL4.3 is characterized by an increase of its IC50 index of 10 times by reference to T5Al virus and 12,5 times by reference to LAI.

V.2—RPR103611 Inhibitor

The increase of the IC50index of the resistant virus LAI-L91H (Labrosse B. et al. J Virol. 2000 vol 74 pages 2142-2150) by reference to RPR103611 has been measured.

The resistant virus LAI-L91H shows an increase of its IC50 index of more than 100 times.

The recombinations obtained by performing the amplification of step (b) with a pair of primers: NEU-A: 5′TAGAAAGAGCAGAAGACAGTGGCAATG3′ (SEQ ID No: 17) and FuB: 5′ GGTGGTAGCTGAAGAGGCACAGG3′ (SEQ ID No: 24), followed by a second amplification with the primers: NEU-C: 5′GTGGGTCACAGTCTATTATGGGG3′ (SEQ ID No: 19) et un mélange des amorces FuD1: 5′TCTGTCTCTCTCTCCACCTTCTTCTT3′ (SEQ ID No: 26) et FuD2: 5′TCTGTCTTGCTCTCCACCTTCTTCTT3′ (SEQ ID No: 27) are also highly efficient.

In fact the control of the viral production allow to verify that in order to obtain a satisfy viral production it's only necessary to use 10-50 ng of the PCR product to carry out the recombination step.

A viralproduction corresponding to 50-400 ng/ml de p24 is obtained after transfection. To infect the target cells, between 0,5 and 8 ng p24/puit (96 well microplates) are used. The increase in optical density obtained after the infection of target cells appears linear if the infection is performed under these conditions.

1. Method for analysing a phenotypic characteristic of HIV viruses present in a biological specimen from a patient, said phenotypic characteristic resulting from one or more mutations of the viral genome liable to influence the viral infection, characterised in that it comprises:
a) the extraction of the nucleic acids contained in said biological specimen,
b) at least one PCR amplification of a segment of the nucleic acids from step (a), each with a pair of primers bordering a nucleic acid sequence of the viral genome liable to comprise at least one mutation,
c) the preparation of a vector comprising the parts of an HIV virus genome required for viral replication except for the segment amplified in step (b) and, if applicable, except for the gene coding for envelope protein,
d) the transfection of a first cell host with:
the nucleic acids contained in step (b),
the vector prepared in step (c),
if applicable, a second vector comprising a gene coding for an envelope protein if the envelope gene is deleted from the vector prepared in step (c),
to obtain a chimeric virus by homologous recombination,
e) the culture of said first cell host under conditions enabling the production of viral particles during a single replication cycle,
f) the infection by the viral particles obtained in step (e) of at least one second cell host liable to be infected by an HIV virus or an HIV pseudotype virus and comprising, if applicable, a marker gene that can only be activated following viral infection,
g) the detection and/or quantification of the marker expressed in step (f) in order to detect at least one characteristic of the HIV viruses present in the biological specimen.

2. Analytical method according to claim 1 characterised in that the PCR amplification in step (b) is carried out with a pair of primers bordering a nucleic acid sequence comprising all or part of a viral genome region selected from: gag, pol, protease, reverse transcriptase, RNAse H, integrase, vif, vpr, tat, rev, vpu, env, nef, cis-active sequences, LTR, dimerisation sequences, splicing regulating sequences or Rev response element (RRE).

3. Analytical method according to any of claims 1 or 2, characterised in that the PCR amplification in step (b) is carried out with a pair of primers bordering a nucleic acid sequence coding for a part of the gag protein of the human immune deficiency virus and a nucleic acid sequence coding for protease, liable to comprise at least one mutation in the gene coding for protease and, in that the vector from step (c) is constructed from an HIV virus genome in which all or part of the gene coding for protease is deleted.

4. Analytical method according to claims 1 to 3 characterised in that the amplification in step (b) comprising at least one mutation in the gene coding for protease is performed with a pair of primers:
(SEQ ID No:1)

| | |
|---|---|
| Fit A-: | (5′ TCA CCT AGA ACT TTA AAT GC 3′) and |

(SEQ ID No:2)

| | |
|---|---|
| Pro A-: | (5′ GGC AAA TAC TGG AGT ATT GTA TG3′3′, |

followed by a second amplification with a pair of primers:
(SEQ ID No:3)

| | |
|---|---|
| Fit B: | (5′ AGA ACT TTA AAT GCA TGG GT 3′) and |

(SEQ ID No:4)

| | |
|---|---|
| Pro B-: | (5′ GGA GTA TTG TAT GGA TTT TCA GG 3′), |

to obtain a DNA segment with 1460 base pairs, ranging from the residues 3950 and 5410 inclusive, and
in that the vector from step (c) is a retroviral vector deleted from the region of the pol reading frame coding for HIV-1 protease ranging from the residues 1505 to 2565 inclusive, deleted from the envelope region and comprising a single MluI restriction site.

5. Analytical method according to claims 1 or 2 characterised in that the PCR amplification in step (b) is carried out with a pair of primers bordering a nucleic acid sequence liable to comprise at least one mutation in the gene coding for reverse transcriptase, and the transfection in step (c) is carried out with a first vector constructed from an HIV virus genome in which all or part of the gene coding for reverse transcriptase is deleted.

6. Analytical method according to claim 1, 2 or 5, characterised in that the amplification in step (b) is performed with a pair of primers:

| | |
|---|---|
| MJ3 | |
| (5′ AGT AGG ACC TAC ACC TGT CA 3′) | (SEQ ID No:5) |
| and | |
| RT-EXT | |
| (5′ TTC CCA ATG CAT ATT GTG AG 3′), | (SEQ ID No:6) |

followed by a second amplification step with a pair of primers:

| | | |
|---|---|---|
| A35 | (5′ TTG GTT GCA TAA ATT TTC CCA TTA GTC CTA TT 3′) and | (SEQ ID No:7) |
| RT-IN | (5′ TTC CCA ATG CAT ATT GTG AG 3′) | (SEQ ID No:8) |

to obtain a DNA segment with 1530 base pairs ranging beyond codon 93 of the region coding for protease and beyond codon 503 of the region coding for polymerase (POL) and
in that the vector from step (c) is a retroviral vector deleted from the region of the pol reading frame coding for HIV-1 reverse transcriptase ranging from the residues 2618 to 2872 inclusive, and comprising a single MluI restriction site.

7. Analytical method according to claims 1, 2, 5 or 6 consisting of determining the susceptibility of an HIV virus to a reverse transcriptase inhibiting compound, characterised in that said reverse transcriptase inhibiting compound is added or not, possibly at different concentrations, to the second cell host, before the infection of said host by the viral particles obtained in step (e), and in that step (g) comprises the comparison of the expression of the marker gene with and without reverse transcriptase inhibiting compound.

8. Analytical method according to claims 1 or 2, characterised in that the PCR amplification in step (b) is carried out with a pair of primers bordering a nucleic acid sequence liable to comprise at least one mutation in the gene coding for integrase, and the vector in step (c) is a retroviral vector in which all or part of the gene coding for integrase is deleted.

9. Analytical method according to claim 1, 2 or 8, characterised in that the amplification in step (b) is performed with the pair of primers:

| | | |
|---|---|---|
| INT | -5′GTTACTAATAGAGGAAGACAAA3′ and | (SEQ ID No:9) |
| B+ | | |
| INT | 5′TTTTGGTGTTATTAATGCT3′, | (SEQ ID No:10) |
| B− | | |

followed by a second amplification step with the pair of primers:

| | | |
|---|---|---|
| INT V+ | 5′CACCCTAACTGACACAACAA3′ and | (SEQ ID No:11) |
| INT V− | 5′AAGGCCTTTCTTATAGCAGA3′, | (SEQ ID No:12) |

to obtain a DNA segment with 1460 base pairs ranging from residues 3950 to 5410 inclusive and
in that the vector from step (c) is a retroviral vector deleted from the entire region of the pol reading frame coding for HIV-1 integrase ranging from the residues 4228 to 5093 inclusive and the region coding for the viral envelope between the positions 6343 and 7611 inclusive.

10. Analytical method according to claims 1, 2, 8 or 9 consisting of determining the susceptibility of an HIV virus to an integrase inhibiting compound, characterised in that said integrase inhibiting compound is added, possibly at different concentrations, during step (e), before step (f) and in that step (g) comprises the comparison of the expression of the marker gene with and without integrase inhibiting compound.

11. Analytical method according to claims 1 or 2, characterised in that the PCR amplification in step (b) is carried out with a pair of primers bordering a nucleic acid sequence liable to comprise at least one mutation in the gene coding for envelope protein, and in that the vector from step (c) is a retroviral vector constructed from an HIV virus genome in which all or part of the gene coding for envelope protein is deleted.

12. Analytical method according to claims 1, 2 or 11 characterised in that the vector from step (c) is a retroviral vector deleted from the entire region coding for the extracellular portion of the gp41 sub-unit of the HIV-1 envelope, ranging from the residues 7745 to 8263 inclusive, the region of the HIV-1 genome forming the Rev response element (RRE).

13. Analytical method according to claims 1, 2, 11 or 12, characterised in that the amplification in step (b) is performed with a pair of primers:
FIN-A: 5′TCAAATATTACAGGGCTGCT3′ (SEQ ID No: 13) and
FIN-B: 5′TAGCTGAAGAGGCACAGG3′ (SEQ ID No: 14) followed by a second amplification step, performed with the pair of primers:

| | | |
|---|---|---|
| FIN-C: | 5′CTATTAACAAGAGATGGTGG3′ and | (SEQ ID No:15) |
| FIN-D: | 5′TCCACCTTCTTCTTCGATT3′, | (SEQ ID No:16) |

to obtain a DNA segment with 965 base pairs ranging from the residues 7553 to 8517 inclusive and
in that the vector in step (c) is a retroviral virus deleted from the entire region coding for the extracellular portion of the gp41 sub-unit of the HIV-1 envelope, ranging from the residues 7745 to 8263 inclusive, and comprises a single MulI restriction site.

14. Analytical method according to claims 1, 2, 11 or 12, characterised in that the amplification in step (b) is performed with a pair of primers:

| | | |
|---|---|---|
| FuA: | 5′AAGCAATGTATGCCCCTCCCAT3′ and | (SEQ ID No:23) |
| FuB: | 5′GGTGGTAGCTGAAGAGGCACAGG3′ | (SEQ ID No:24) |

followed by a second amplification step, performed with the primer:
FuC: 5′ATATGAGGGACAATTGGAGAAGTGA3′ (SEQ ID No: 25) and a mixture of the following primers:

| | |
|---|---|
| FuD1: | |
| 5′TCTGTCTCTCTCTCCACCTTCTTCTT3′ | (SEQ ID No:26) |
| FuD2: | |
| 5′TCTGTCTTGCTCTCCACCTTCTTCTT3′, | (SEQ ID No:27) |

mixture being preferently carried out in a ratio comprised between (10%:90%) and (90%:10%) more preferently between (60%:40%) and (40%:60%),
to obtain a DNA segment with 805 base pairs ranging from the residues 7635 to 8440 inclusive and the vector in step c is a retroviral virus deleted from the entire region coding for the extracellular portion of the gp41 sub-unit of the HIV-1 envelope, ranging from the residues 7745 to 8263 inclusive, and comprises a single MulI restriction site.

15. Analytical method according to claims 1, 2, 11 or 12, characterised in that the amplification in step (b) is performed with a pair of primers:

| | |
|---|---|
| NEU-A: | |
| 5′TAGAAAGAGCAGAAGACAGTGGCAATG3′ and | (SEQ ID No:17) |
| FIN-B: | |
| 5′TAGCTGAAGAGGCACAGG3′ | (SEQ ID No:14) |

second amplification step, performed with the pair of primers:

| | | |
|---|---|---|
| NEU-C: | 5′GTGGGTCACAGTCTATTATGGGG3′ | (SEQ ID No:18) |
| and | | |
| FIN-D: | 5′TCCACCTTCTTCTTCGATT3′, | (SEQ ID No:16) |

to obtain a DNA segment with 2320 base pairs ranging from the residues 6197 to 8517 inclusive and
in that the vector in step (c) is a retroviral vector deleted from the entire region coding for the majority of the gp120 sub-unit and the extracellular portion of the gp41 sub-unit of the HIV-1 envelope, ranging from the residues 6480 to 8263 inclusive, and comprises a single MulI restriction site.

16. Analytical method according to claims 1, 2, 11 or 12, characterised in that the amplification in step (b) is performed with a pair of primers:

| | |
|---|---|
| NEU-A: | |
| 5′TAGAAAGAGCAGAAGACAGTGGCAATG3′ and | (SEQ ID No:17) |
| FuB: | |
| 5′GGTGGTAGCTGAAGAGGCACAGG3′, | (SEQ ID No:24) |

followed by a second amplification step, performed with the pair of primers:
NEU-C: 5′GTGGGTCACAGTCTATTATGGGG3′ (SEQ ID No: 18) and a mixture of the following primers

| | |
|---|---|
| FuD1: | |
| 5′TCTGTCTCTCTCTCCACCTTCTTCTT3′ and | (SEQ ID No:26) |
| FuD2: | |
| 5′TCTGTCTTGCTCTCCACCTTCTTCTT3′, | (SEQ ID No:27) |

mixture being preferently carried out in a ratio comprised between (10%:90%) and (90%:10%) more preferently between (60%:40%) and (40%:60%),
to obtain a DNA segment with 2118 base pairs ranging from the residues 6322 to 8440 inclusive and the vector in step c is a retroviral vector deleted from the entire region coding for the majority of the gp120 sub-unit and the extracellular portion of the gp41 sub-unit of the HIV-1 envelope, ranging from the residues 6480 to 8263 inclusive, and comprises a single MulI restriction site.

17. Analytical method according to claims 1, 2, 11 or 12, characterised in that the amplification in step (b) is performed with a pair of primers:

| | |
|---|---|
| E00: | |
| 5′TAGAAAGAGCAGAAGACAGTGGCAATGA3′ and | (SEQ ID No:19) |
| ES8B: | |
| 5′CACTTCTCCAATTGTCCCTCA3′, | (SEQ ID No:22) |

followed by a second amplification step, performed with the pair of primers:

| | |
|---|---|
| E20: | |
| 5′GGGCCACACATGCCTGTGTACCCACAG3′ and | (SEQ ID No:21) |
| E115: | |
| 5′AGAAAAATTCCCCTCCACAATTAA3′, | (SEQ ID No:22) |

to obtain a DNA segment with 938 base pairs ranging from the residues 6426 to 7364 inclusive and
in that the vector in step (c) is a retroviral vector deleted from the region, coding for the domains ranging from the loop V1 to the loop V3 of the HIV-1 envelope ranging from 6617 to 7250 inclusive and comprises a single NheI restriction site.

18. Analytical method according to claims 1, 2, 11 to 17 consisting of determining the susceptibility of an HIV virus to a fusion inhibiting compound targeting HIV-1 gp41 protein, characterised in that said fusion inhibiting compound is added, possibly at different concentrations, during the culture of the cell host obtained in step (e), before step (f) and in that step (g) comprises the comparison of the expression of the marker gene with and without fusion inhibiting compound targeting HIV-1 gp41.

19. Analytical method according to claims 1, 2, 11, 12 or 15 consisting of determining the susceptibility of an HIV virus to a compound inhibiting the entry of said HIV virus into a target cell, characterised in that said entry inhibiting compound is added, possibly at different concentrations, to the cell host obtained in step (e) before the infection in step (f) and in that step (g) comprises the comparison of the expression of the marker gene with and without entry inhibiting compound.

20. Analytical method according to claims 1, 2, 11, 12 or 15 consisting of determining the susceptibility of an HIV virus to the inhibitory action of antibodies, characterised in that said method is carried out, firstly without antibodies and, secondly, with the antibody, possibly at different concentrations, said antibody being present in step (e), and in that step (g) comprises the comparison of the expression of the marker gene with and without antibodies.

21. Analytical method according to claims 1, 2, 11, 12 or 15 consisting of determining the tropism of an HIV virus for a cell receptor, characterised in that the infection in step (f) with the viral particles obtained in step (e) is performed on two separate cell hosts and step (g) comprises the comparison of the expression of the marker gene by each of the two separate cell hosts.

22. Analytical method according to claim 21 characterised in that one of two cell hosts infected in step (g) expresses the CCR5 receptor and the other expresses the CXCR4 receptor.

23. Analytical method according to claims 1, 2, 11, 12 or 15 consisting of determining the susceptibility of an HIV virus to an inhibiting compound targeting HIV-1 co-receptors, characterised in that said inhibiting compound targeting HIV-1 co-receptors is added or not, possibly at different concentrations, during the culture step (e), in that the infection in step (f) is performed on two separate cell hosts and in that step (g) comprises the comparison of the expression of the marker gene by each of the two separate cell hosts.

24. Analytical method according to claims 1, 2, 11, 12 or 17 consisting of analysing the tropism of an HIV virus for a cell receptor, characterised in that the infection in step (f) with the viral particles obtained in step (e) is performed on two separate cell hosts and step (g) comprises a comparison of the expression of the marker gene by each of the two separate cell hosts.

25. Analytical method according to claims 1, 2, 11, 12 or 17 consisting of analysing the susceptibility of an HIV virus to an inhibiting compound targeting HIV-1 co-receptors, characterised in that said inhibiting compound targeting HIV-1 co-receptors is added, possibly at different concentrations, during the culture in step (d), in that the infection in step (i) with the viral particles in step (e) is performed on two separate cell hosts and in that step (g) comprises the comparison of the expression of the marker gene by each of the two separate cell hosts.

26. Analytical method according to any of claims 1 to 17 consisting of determining the infectivity or replicative capacity of an HIV virus characterised in that step (g) comprises the comparison of the expression of the marker gene by the second cell host infected with the viral particles obtained by applying steps (a) to (f) to a biological specimen from a patient, and the expression of the marker gene by the same second cell host infected with the reference viral particles obtained by applying steps (a) to (f) to a specimen containing a reference virus.

27. Analytical method according to claim 26 characterised in that the reference viral particles from a reference virus are viral particles obtained by applying steps (a) to (f) to a biological specimen from the same patient at an earlier stage or treatment or before said treatment.

28. Analytical method according to claims 1 to 17 consisting of determining the susceptibility of an HIV virus to hydroxyurea, characterised in that hydroxyurea is added or not, possibly at different concentrations, either during the culture step (e), or to the second cell host, before the infection of said host in step (f) and in that step (g) comprises the comparison of the expression of the marker gene with and without hydroxyurea.

29. Analytical method according to any of claims 1 to 28 characterised in that the culture step (e) is performed during a period ranging from 12 hours to 72 hours, preferentially from 24 hours to 48 hours.

30. A kit for implementing the method according to any of claims 1 to 29 characterised in that it comprises:
i) a pair of primers bordering a nucleic acid sequence of the viral genome liable to comprise at least one mutation,
ii) a vector comprising the parts of an HIV virus genome required for viral replication except for the segment amplified with the primers defined in (i) and the gene coding for the envelope protein,
iii) a second vector comprising a gene coding for envelope protein,
iv) a first cell host liable to be infected by an HIV virus,
v) a second cell host liable to be infected by an HIV virus and comprising a marker gene that can only be activated following viral infection,
vi) the products and reagents required to carry out PCR amplification,
vii) the products and reagents used to detect the expressed marker.

31. A kit according to claim 30, characterised in that it comprises:
i) the sequence primer pairs:
SEQ ID No:1 and SEQ ID No:2
SEQ ID No:3 and SEQ ID No:4
ii) a retroviral vector deleted from the region of the pol reading frame coding for HIV-1 protease ranging from the residues 1505 to 2565 inclusive, deleted from the envelope region and comprising a single MluI restriction site,
iii) a pseudotype virus with a gene coding for an envelope protein,
iv) a first cell host liable to be infected by an HIV virus,
v) a second cell host liable to be infected by an HIV virus and comprising a marker gene that can only be activated following viral infection,
vi) the products and reagents required to carry out PCR amplification,
vii) the products and reagents used to detect the expressed marker.

32. A kit according to claim 30, characterised in that it comprises:
i) the sequence primer pairs:
SEQ ID No:5 and SEQ ID No:7
SEQ ID No:6 and SEQ ID No:8
ii) a retroviral vector deleted from the region of the pol reading frame coding for HIV-1 reverse transcriptase ranging from the residues 2618 to 2872 inclusive, and comprising a single MluI restriction site,
iii) a pseudotype virus with a gene coding for an envelope protein,
iv) a first cell host liable to be infected by an HIV virus,
v) a second cell host liable to be infected by an HIV virus and comprising a marker gene that can only be activated following viral infection,
vi) the products and reagents required to carry out PCR amplification,
vii) the products and reagents used to detect the expressed marker.

33. A kit according to claim 30 characterised in that it comprises:
i) the sequence primer pairs:
SEQ ID No:9 and SEQ ID No:10
SEQ ID No:11 and SEQ ID No:12
ii) a retroviral vector deleted from the region of the pol reading frame coding for HIV-1 integrase ranging from the residues 4228 to 5093 inclusive and the region coding for the viral envelope between the positions 6343 and 7611 inclusive,
iii) a pseudotype virus with a gene coding for an envelope protein,
iv) a first cell host liable to be infected by an HIV virus,
v) a second cell host liable to be infected by an HIV virus and comprising a marker gene that can only be activated following viral infection,
vi) the products and reagents required to carry out PCR amplification,
vii) the products and. reagents used to detect the expressed marker.

34. A kit according to claim 30, characterised in that it comprises:
i) the sequence primer pairs:
SEQ ID No:13 and SEQ ID No:14
SEQ ID No:15 and SEQ ID No:16
ii) a retroviral vector deleted from the entire region coding for the extracellular portion of the HIV-1 envelope gp41 sub-unit, ranging from the residues 7745 to 8263 inclusive, and comprising a single MluI restriction site,
iv) a first cell host liable to be infected by an HIV virus,
v) a second cell host liable to be infected by an HIV virus and comprising a marker gene that can only be activated following viral infection,
vi) the products and reagents required to carry out PCR amplification,
vii) the products and reagents used to detect the expressed marker.

35. A kit according to claim 30, characterised in that it comprises:
i) the sequence primers:
(SEQ ID No: 23) and (SEQ ID No: 24)
(SEQ ID No: 25) and the mixture of primers (SEQ ID No: 26) and (SEQ ID No: 27),
ii) a retroviral vector deleted from the entire region coding for the extracellular portion of the HIV-1 envelope gp41 sub-unit, ranging from the residues 7745 to 8263 inclusive, and comprising a single MluI restriction site,
iv) a first cell host liable to be infected by an HIV virus,
v) a second cell host liable to be infected by an HIV virus and comprising a marker gene that can only be activated following viral infection,
vi) the products and reagents required to carry out PCR amplification,
vii) the products and reagents used to detect the expressed marker.

36. A kit according to claim 30, characterised in that it comprises:
i) the sequence primer pairs:
SEQ ID No:17 and SEQ ID No:14
SEQ ID No:18 and SEQ ID No:16
ii) a retroviral vector deleted from the entire region coding for the majority of the gp120 sub-unit and the extracellular portion of the HIV-1 envelope gp41 sub-unit, ranging from the residues 6480 to 8263 inclusive, and comprising a single MulI restriction site,
iv) a first cell host liable to be infected by an HIV virus,
v) a second cell host liable to be infected by an HIV virus and comprising a marker gene that can only be activated by viral particles,
vi) the products and reagents required to carry out PCR amplification,
vii) the products and reagents used to detect the expressed marker.

37. A kit according to claim 30, characterised in that it comprises:
i) the sequence primer pairs:
SEQ ID No:17 and SEQ ID No:24
SEQ ID No:18 and SEQ ID No:26 and SEQ ID No:27
ii) a retroviral vector deleted from the entire region coding for the majority of the gp120 sub-unit and the extracellular portion of the HIV-1 envelope gp41 sub-unit, ranging from the residues 6480 to 8263 inclusive, and comprising a single MulI restriction site,
iv) a first cell host liable to be infected by an HIV virus,
v) a second cell host liable to be infected by an HIV virus and comprising a marker gene that can only be activated by viral particles,
vi) the products and reagents required to carry out PCR amplification,
vii) the products and reagents used to detect the expressed marker

38. A kit according to claim 30, characterised in that it comprises:
i) the sequence primer pairs:
SEQ ID No:19 and SEQ ID No:20
SEQ ID No:21 and SEQ ID No:22
ii) a retroviral vector deleted from the region, coding for the domains ranging from the loop V1 to the loop V3 of the HIV-1 envelope, ranging from 6617 to 7250 inclusive, and comprising a single NheI restriction site,
iv) a first cell host liable to be infected by an HIV virus,
v) a second cell host liable to be infected by an HIV virus and comprising a marker gene that can only be activated following viral infection,
vi) the products and reagents required to carry out PCR amplification,
vii) the products and reagents used to detect the expressed marker.


SEQUENCE LISTING (27 sequences)

SEQ ID NO: 1 - DNA, 20 bp, Artificial Sequence
  Fit-A sequence.  Amplifies a region coding for protease.
tcacctagaactttaaatgc

SEQ ID NO: 2 - DNA, 23 bp, Artificial Sequence
  Pro A sequence. Amplifies a region coding for protease.
ggcaaatactggagtattgtatg

SEQ ID NO: 3 - DNA, 20 bp, Artificial Sequence
  Fit B sequence. Amplifies a region coding for protease.
agaactttaaatgcatgggt

SEQ ID NO: 4 - DNA, 23 bp, Artificial Sequence
  Pro B sequence. Amplifies a region coding for protease.
ggagtattgtatggattttcagg

SEQ ID NO: 5 - DNA, 20 bp, Artificial Sequence
  MJ33 sequence. Amplifies a region coding for reverse transcriptase.
agtaggacctacacctgtca

SEQ ID NO: 6 - DNA, 20 bp, Artificial Sequence
  RT-EXT sequence. Amplifies a region coding for reverse transcriptase.
ttcccaatgcatattgtgag

SEQ ID NO: 7 - DNA, 32 bp, Artificial Sequence
  A35 sequence. Amplifies a region coding for reverse transcriptase.
ttggttgcataaattttcccattagtcctatt

SEQ ID NO: 8 - DNA, 20 bp, Artificial Sequence
  RT-IN sequence. Amplifies a region coding for reverse transcriptase.
ttcccaatgcatattgtgag

SEQ ID NO: 9 - DNA, 22 bp, Artificial Sequence
  INT B+ sequence. Amplifies a region coding for integrase.
gttactaatagaggaagacaaa

SEQ ID NO: 10 - DNA, 19 bp, Artificial Sequence
  INT B- sequence. Amplifies a region coding for integrase.
ttttggtgttattaatgct

SEQ ID NO: 11 - DNA, 20 bp, Artificial Sequence
  INT V+ sequence. Amplifies a region coding for integrase.
caccctaactgacacaacaa

SEQ ID NO: 12 - DNA, 20 bp, Artificial Sequence
  INT V- sequence. Amplifies a region coding for integrase.
aaggcctttcttatagcaga

SEQ ID NO: 13 - DNA, 20 bp, Artificial Sequence
  FIN-A sequence. Amplifies a region coding for envelope gene.
tcaaatattacagggctgct

SEQ ID NO: 14 - DNA, 18 bp, Artificial Sequence
  FIN-B sequence. Amplifies a region coding for envelope gene.
tagctgaagaggcacagg

SEQ ID NO: 15 - DNA, 20 bp, Artificial Sequence
  FIN-C sequence. Amplifies a region coding for envelope gene.
ctattaacaagagatggtgg

SEQ ID NO: 16 - DNA, 19 bp, Artificial Sequence
  FIN-D sequence.  Amplifies a region coding for envelope gene.
tccaccttcttcttcgatt

SEQ ID NO: 17 - DNA, 27 bp, Artificial Sequence
  NEU-A sequence. Amplifies a region coding for envelope gene.
tagaaagagcagaagacagtggcaatg

SEQ ID NO: 18 - DNA, 23 bp, Artificial Sequence
  NEU-C sequence. Amplifies a region coding for envelope gene.
gtgggtcacagtctattatgggg

SEQ ID NO: 19 - DNA, 28 bp, Artificial Sequence
  E00 sequence. Amplifies a region coding for envelope gene.
tagaaagagcagaagacagtggcaatga

SEQ ID NO: 20 - DNA, 21 bp, Artificial Sequence
  ES8B sequence. Amplifies a region coding for envelope gene.
cacttctccaattgtccctca

SEQ ID NO: 21 - DNA, 27 bp, Artificial Sequence
  E20 sequence. Amplifies a region coding for envelope gene.
gggccacacatgcctgtgtacccacag

SEQ ID NO: 22 - DNA, 24 bp, Artificial Sequence
  E115 sequence. Amplifies a region coding for envelope gene.
agaaaaattcccctccacaattaa

SEQ ID NO: 23 - DNA, 22 bp, Artificial Sequence
  FuA sequence. Amplifies a region coding for envelope gene.
aagcaatgtatgcccctcccat

SEQ ID NO: 24 - DNA, 23 bp, Artificial Sequence
  FuB sequence. Amplifies a region coding for envelope gene.
ggtggtagctgaagaggcacagg

SEQ ID NO: 25 - DNA, 25 bp, Artificial Sequence
  FuC sequence. Amplifies a region coding for envelope gene.
atatgagggacaattggagaagtga

SEQ ID NO: 26 - DNA, 26 bp, Artificial Sequence
  FuD1 sequence. Amplifies a region coding for envelope gene.
tctgtctctctctccaccttcttctt

SEQ ID NO: 27 - DNA, 26 bp, Artificial Sequence
  FuD2 sequence. Amplifies a region coding for envelope gene.
tctgtcttgctctccaccttcttctt